(19) United States
(12) Patent Application Publication
(10) Pub. No.: US 2015/0353967 A1
(43) Pub. Date: Dec. 10, 2015

(54) METHOD FOR INCREASED PRODUCTIVITY OF POLYHYDROXYALKANOATES (PHAS) IN FED-BATCH PROCESSES FOR BIOMASS DERIVED FROM THE TREATMENT OF WASTEWATER

(71) Applicants: Alan Gideon WERKER, Lomma (SE); Fernando MORGAN-SAGASTUME, Malmö (SE); Lamija KARABEGOVIC, Helsingborg (SE); Simon Olof Harald BENGTSSON, Dalby (SE); Francesco VALENTINO, Roma (IT); Mauro MAJONE, Roma (IT); VEOLIA WATER SOLUTIONS & TECHNOLOGIES sUPPORT, Saint-Maurice (FR)

(72) Inventors: Alan Gideon WERKER, Lomma (SE); Fernando MORGAN-SAGASTUME, Malmo (SE); Lamija KARABEGOVIC, Helsingborg (SE); Simon Olof Harald BENGTSSON, Dalby (SE); Francesco VALENTINO, Roma (IT); Mauro MAJONE, Roma (IT)

(73) Assignee: Veolia Water Solutions & Technologies Support, Saint-Maurice (FR)

(21) Appl. No.: 14/760,217

(22) PCT Filed: Jan. 13, 2014
(86) PCT No.: PCT/IB2014/058242

§ 371 Date: Jul. 10, 2015

Related U.S. Application Data
(60) Provisional application No. 61/751,449, filed on Jan. 11, 2013.

(51) Int. Cl. C12P 7/62; C02F 3/12

(52) U.S. Cl. CPC C12P 7/625; C02F 3/12

(57) ABSTRACT

This disclosure relates to methods for producing a PHA-rich-biomass from open mixed cultures. Mixed liquor containing biomass is directed into a fed-batch reactor. The reactor includes at least one biomass stimulating zone and at least one biomass maintenance zone. A feed containing RBCOD, bioavailable N, and bioavailable P is directed into the fed-batch reactor. The respiration rate of at least a portion of the biomass is intermittently and repeatedly stimulated in the stimulating zone by directing the feed into the fed-batch reactor and exposing the biomass to a relatively high concentration of RBCOD. The biomass is then transferred to the maintenance zone, where the biomass is exposed to a relatively low concentration of RBCOD. Thereafter, the biomass is circulated back and forth between the stimulating zone and the maintenance zone. Throughout the methods, the concentration of N and/or P relative to the RBCOD in the feed is controlled by controlling the ratio of N to RBCOD and/or the ration of P to RBCOD in the feed.

### RELATED APPLICATIONS

This application claims priority under 35 U.S.C. §119(e) from the following U.S. provisional application: Application Ser. No. 61/751,449 filed on Jan. 11, 2013. That application is incorporated in its entirety by reference herein.

### BACKGROUND

Biological treatment of wastewaters for removal of the chemical oxygen demand (COD) produces a biomass. Wasted biomass represents a solid waste disposal problem. One opportunity that has attracted much interest is the production of biodegradable polymers by biomass, such as activated sludge from treating wastewater. In this way, waste sludge produced becomes instead a valuable by-product that can be harvested from the treatment process.

It is known that biomass treating wastewater can be harvested and made to accumulate polyhydroxyalkanoates (PHAs), a group of polyesters naturally produced by some bacteria as intermediate carbon and energy reservoirs. PHAs are biopolymers that can be recovered from biomass and converted into biodegradable plastics of commercial value that are useful for a broad range of practical applications (see for examples, US 2010/0200498, WO 2011/070544A2, WO 2011/073744A1, WO 2012/022998A1, WO 2012/023114A1).

Embodiments to condition the thermal stability of the PHA-In-biomass so as to maintain the PHA molecular weight and improve the product quality (value) during the PHA recovery from the biomass have been disclosed in WO 2012/022998A1. In combination, the above-cited disclosures provide for a recipe to integrate services of pollution control and residuals management with the production of a biodegradable and bio-based polymer of known practical and economic value. Based on the aforementioned disclosures, pollution control facilities can already today, and in many instances without dramatic modifications, be converted such that the economic burden of sludge disposal is offset into to a benefit of PHA-rich biomass production.

Effectively wasted biomass, such as excess activated sludge, coming from biological treatment systems is converted from a process residual into a value-added raw product, namely a PHA-rich-biomass, by disposing this biomass to a PHA accumulation process whereby readily biodegradable COD (RBCOD), such as volatile fatty acids as the principal organic substrate, is fed to the wasted (harvested) biomass in a controlled manner. Embodiments to accumulate PHA in a biomass and produce a PHA-rich biomass containing PHA with high molecular weight are described in WO 2011/070544A2.

The application of the methods and processes described in WO 2011/070544A2 generally permit for the accumulation of PHA in a mixed culture of biomass. By mixed culture it is meant a biomass comprising a mixture of more than one type of population of specie of bacteria. The biomass is generally anticipated to be enriched with populations of species of bacteria that can convert RBCOD to PHA. Notwithstanding any such enrichment, the mixed culture biomass will also contain other species of bacteria in the biomass that will not store PHA and, if stimulated, they will consume RBCOD to produce non-PHA containing biomass. Preferred accumulation process conditions are lost if the rate of production of PHA mass by the biomass becomes less than the rate of production of non-PHA biomass.

In WO 2011/070544A2 it is taught how stimulating and maintenance process zones can be used to sustain a feed-on-demand process for an industrial scale mixed culture PHA accumulation. Maintenance zones generally maintain the respiring biomass within an environment of relatively low RBCOD concentration. Stimulating zones generally expose fractions of the biomass at any given time to relatively high RBCOD concentrations and in so doing work to stimulate the overall process biomass to a high level of respiration due, to a large extent, to the metabolic processes of converting RBCOD into stored PHA. This overall high biomass respiration rate drives the demand for substrate and controlling the substrate supply based on maintaining this high biomass respiration rate thereby can establish the process control strategy of “feed-on-demand.” Since the biomass is otherwise maintained in zones of relatively low RBCOD concentration, there exists a limitation of available substrate. Non-PHA biomass production, that is to say biomass growth by cell-division, exhibits a respiration rate that generally is proportional to the substrate concentration when the substrate concentration is relatively low as described for example the Monod Equation. By controlling the substrate supply so as to maintain relatively low substrate levels in the maintenance zone, the level of non-PHA biomass production can be mitigated by giving preference to the respiration and RBCOD consumption by the PHA storing bacteria in the process.

We find that the respiration and activity of PHA storing bacteria can be sustained by periodically and repeatedly subjecting the biomass to conditions of “feast” in a stimulating zone that is separate from the maintenance zone. The conditions of this feast stimulation do not need to be aerobic but a sufficient, albeit brief, time of exposure seems to be desirable. In practice one would like to keep this period of stimulation relatively short so as to limit the onset of increased respiration of the non-PHA storing bacteria in the process. Notwithstanding the possibility for other interpretations of process and future developments of insight into the biomass respiration, we find that generally the requisite time to reach a full stimulation to a high respiration rate from low levels of endogenous respiration and for a PHA storing biomass is of a time scale order of magnitude of less than one minute.

### SUMMARY OF THE INVENTION

The methods herein relate to producing a PHA-rich-biomass from open cultures. Mixed liquor containing biomass is directed into a fed-batch reactor. The reactor includes at least one biomass stimulating zone and at least one biomass maintenance zone. A feed is provided that contains biodegradable chemical oxygen demand (RBCOD), bio-available nitrogen (N), and bio-available phosphorus (P). The concentrations of the bio-available N and P in the feed are adjusted relative to the RBCOD such that the average N to RBCOD ratio is between 2 mg-N/g-RBCOD and 15 mg-N/g/RBCOD and the average P to RBCOD ratio is between 0.5 mg-P/g-RBCOD and 3 mg-P/g-RBCOD. A fraction of the biomass in the reactor is exposed to the adjusted feed in the stimulating zone. This stimulates the biomass respiration rate. The adjusted feed is provided such that the average respiration rate of the stimulated biomass is greater than 50% of the biomass extant maximum respiration rate. The fraction of the biomass that was exposed to the adjusted feed is then transferred to the maintenance zone. In the maintenance zone, the average RBCOD concentration is maintained at less than half the average concentration of RBCOD in the stimulation zone. The mixed liquor containing biomass is circulated between the stimulating zone and the maintenance zone. This results in fractions of biomass being repeatedly exposed to the feed and reaching a high respiration rate when in the stimulating zone, while maintaining the biomass fractions at this elevated respiration rate even at a low RBCOD concentration when in the maintenance zone.

The methods further relate to a fed-batch process for producing PHA in biomass from open mixed cultures by supplying substrate in such a way so as to promote for PHA storage in the biomass along with concurrent growth of non-PHA biomass, at least for some period of time during the fed batch process, whereby:
a. the fed-batch process is sustained over a period of time with an increasing or steady level of PHA content in the biomass;
b. the steady level of PHA content reached In the biomass is greater than 0.40 g-PHA/g-VSS, preferably greater than 0.50 g-PHA/g-VSS, and most preferably greater than 0.60 g-PHA/g-VSS;
c. readily biodegradable organic substrate or readily biodegradable chemical oxygen demand (RBCOD) is supplied together with bioavailable nitrogen (N) and phosphorus (P) with a N/COD ratio on average within the range of 2 to 15 mg-N/g-RBCOD, and a P/COD ratio on average within the range of 0.5 to 3 mg-P/g-RBCOD;
d. the biomass as a whole, or in part, is repeatedly stimulated so as to be sustained on average above 50 and preferably above 70 percent maximum extant respiration rate, while being otherwise maintained under conditions of average carbon substrate concentration below 100 and preferably below 10 mg-RBCOD/L;
e. fractions of the biomass at any one time are circulated to a zone of elevated substrate concentration and are stimulated to a maximum extant respiration rate with exposure to peak average organic substrate concentrations above 100 mg-RBCOD/L but preferably less than 500 mg-RBCOD/L and most preferably less than 2000 mg-RBCOD/L;
f. the initial content of PHA in the biomass supplied to the fed-batch process is less than 0.10 g-PHA/g-VSS, preferably less than 0.05 g-PHA/g-VSS and most preferably less than 0.02 g-PHA/g-VSS;
g. the initial concentration of biomass in the process is greater than 500 mg-VSS/L, but preferably greater than 1000 mg-VSS/L and most preferably greater than 2000 mg-VSS/L; and

### DESCRIPTION OF THE DRAWINGS

FIG. 1 is a graph showing PHA accumulation potential (PAP) (g-PHA/g-VSS) for FWP as RBCOD and Acetate as RBCOD.

FIG. 2 is a graph showing PHA content (g-PHA/g-VSS) and Mass Produced or Consumed (g) over time (hours) for Acetate as RBCOD.

FIG. 3 is a graph showing PHA content (g-PHA/g-VSS) and Mass Produced or Consumed (g) over time (hours) for FWP as RBCOD.

FIG. 4 is a graph showing biomass yield on RBCOD (g-COD/g-COD) for FWP as RBCOD and Acetate as RBCOD.

FIG. 5 is a graph showing PHA Accumulation Potential (PAP) (g-PHA/g-VSS) versus relative initial mass (g/g) of biomass inorganic content and PHA.

FIG. 6 is a graph showing N/COD or P/COD versus the percent N or P consumed,

FIG. 7 is a graph showing the N/COD ratio (mg N/g COD) versus relative mass increase (g/g).

FIG. 8 is a graph showing the P/COD ratio (mg P/g COD) versus relative mass increase (g/g).

FIG. 9 is a schematic illustration showing embodiments for implementing the invention in a wastewater treatment process.

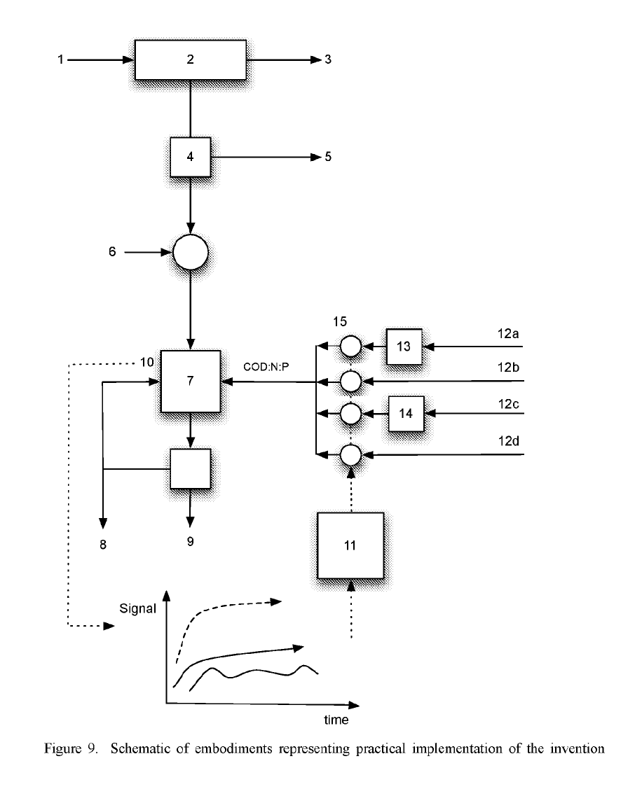

FIG. 10 is a schematic illustration

FIG. 11 is a flow diagram showing a control schematic for controlling the feed and concentration of various constituents to a fed-batch reactor of a PHA accumulation system.

FIG. 12 is a schematic illustration of a control system for controlling the feed and the concentration of various constituents to a fed-batch reactor of a PHA accumulation system.

### DETAILED DESCRIPTION OF THE INVENTION

Wastewater biological treatment and PHA production in mixed cultures has been demonstrated as a two-stage undertaking.

In the first stage, biomass is produced (BiPP—Biomass Production Process) while providing a service of water quality amelioration in a way so as to enrich the biomass with a significant PHA-storage potential. One can refer to disclosures of examples in the art including US 2010/0200498, WO 2010/073744A1 and WO 2012/023114A1. Biomass harvested from Me BiPP is utilized as a resource for PHA production. Generally, the biomass harvested from the BiPP is with a negligible PHA content. The PHA content from this harvested biomass should be less than 10% of the dry biomass weight (g-PHA/g-VSS), but more preferably less than 5% and even more preferably less than 2%.

In the second stage, harvested surplus biomass from the BiPP is utilized in a fed-batch PHA production process (PPP). The objective of the PPP is to achieve a high degree of PHA accumulation in this biomass. The PHA content of the biomass should be higher than 40% of the organic dry solids content (g-PHA/g-VSS), but preferably higher than 50%, and even more preferably higher than 60%. The high PHA content of the biomass should be achieved in as short a period as possible. In general the PHA accumulation process to reach a maximum PHA content in the biomass should take less than 48 hours, preferably less than 24 hours, and even more preferably less than 12 hours.

It is important to recognize the difference between the extant PHA production rate in the PPP and the average PHA production rate. In nutrient starved PHA production processes, the extant PHA production rate is generally higher in the beginning and negligible some hours later as the biomass reaches its PHA accumulation potential (PAP). However, we find that the extant PHA production rate can be sustained and a higher average production rate when enough nutrients are provided in combination with high respiration rates and restricted availability of COD. If nutrients are supplied in the right amounts, a growth of PHA storing organisms and PHA storage can be sustained whereby an extant PAP is exhibited by the biomass, and, at the same time, the overall active biomass content increases in time. The increased average PHA accumulation rates, coupled to a concurrent increase in the active biomass accumulating PHA in the PPP subject to a constrained process time, will result in an improvement in the process productivity. Therefore, in practice, due to addition of sufficient additional nutrients to the process other than RBCOD, the PPP can be operated productively tong after the maximum PAP of the biomass has been reached. Consequently, fed-batch mixed culture PHA accumulation processes can be operated, for purposes of increased productivity, for much longer periods of time. In order to avoid the accumulation of a too high biomass concentration in the process, PHA-rich-biomass can be harvested progressively over time rather than just at the point in time of the batch process termination. In general the PHA accumulation process may be operated for greater than 12 hours, preferably greater than 24 hours, and even more preferably greater than 48 hours so long as the PHA content of the biomass is sustained with concurrent non-PHA biomass production. Constraints on the time available for the PPP are tied to the supply rate of feedstocks, containing RBCOD, N and P, and of harvested biomass from the BiPP.

PHA storage is promoted by a supply rate of COD whereby the substrate is supplied on demand at a feed rate and a means of application that sustains a high respiration rate, preferentially by those microorganisms prone to assimilate carbon more rapidly as PHA. As demonstrated herein, those microorganisms that are stimulated into maximal respiration of PHA storage at the beginning of the PPP are also likely to increase in active biomass concurrently with PHA storage activity. This is shown when the biomass increases while the rate increase in PHA content of the biomass is greater than or equal to zero.

Experimental results discussed herein suggest that those microorganisms in harvested BiPP biomass that are able to store PHA can be encouraged to maintain a kinetic competitive advantage in the COD assimilation. Selected restricted nutrient (N, P) supply below the levels for just active biomass growth and above levels of nutrient starvation, along with a COD supply, results in robust, sustained active biomass growth with PHA production in the PPP beyond the growth and production afforded by prior methods. The strategy is to generate metabolic stimulation of the biomass in the PPP, such that the fraction of the biomass storing PHA maintains a selective advantage for the utilization of nutrients for its own growth for as long as possible in the fed-batch process operation. Such an advantage can be fostered in PPPs if nutrients are supplied under conditions of restricted carbon substrate availability ensuring maximized biomass respiration, thus promoting biomass growth with increased time averaged PHA storage yields. Under such conditions of combined growth and storage, it is possible to drive the PHA storage process and to operate PPPs so as to achieve a greater final mass of product from the fed-batch system.

In general, under nutrient availability with readily available carbon substrate, growth will tend to become favoured compared to storage, whereas, under nutrient restriction, a higher fraction of the substrate may be driven towards storage and growth of the biomass will become constrained. For example, it is observed that under ammonium-limiting conditions, the growth yield increases proportionally to increased ammonium concentrations while the storage yield decreases (Serafim et al. 2004. Biotechnology and Bioengineering, 87:145-180.). If growth in excess of storage occurs during a PHA batch accumulation, PHA content in the biomass will reach a maximum value from which it will decrease since the preferential growth of non-PHA biomass becomes dominant over storage of PHAs (Bengtsson at al. 2008. Bioresource Technology, 99:509-516.). Thus, the present disclosure concerns the optimization of nutrient and substrate supply for the purpose of achieving conditions for achieving an increased productivity in mass of PHA derived from a batch mixed culture PHA production process.

A number of academic studies have been conducted with PPPs, in which an influence of supplied N and P levels on the biomass PHA accumulation potential (PAP) has been examined. (For example see Dionisi D et al. 2004. Biotechnology and Bioengineering, 85: 569-579, Dionisi D et al. 2005. Journal of Chemical Technology and Biotechnology, 80:1305-1318, Dionisi D et al. 2006. Biotechnology and Bioengineering, 93(1): 76-88. Bengtsson S. et al. 2008 Bioresource Technology, 99:509-516. Johnson et al. 2010. Water Research, 44:2141-2152. Wan et al. 2010. Journal of Environmental Sciences, 22(10)1602-1607. Basak et al. 2011. Bioprocess and Biosystems Engineering, 34(8):1007-1016). Notwithstanding the reviewed findings of experimentation of the use of nutrients in PPPs, these studies have been primarily focused on the influence of nutrients on the expressed biomass PAP and not on the PPP productivity with respect to reliably driving a stable process of combined PHA storage with active biomass growth. Know-how has been lacking with respect to understanding the combination of a selected range of N and P supply to a biomass towards the control of fed-batch PPPs for a sustained activity of PHA storage, with the result of an increase in the process productivity with respect to the mass of PHA produced by the biomass supplied to the PPP.

The methods described herein relate to a means to increase the productivity of PHA-rich biomass production from biomass harvested from biological treatment systems (Example 3). A means to improve such productivity is to achieve an increase in the mass of PHA generated per unit volume and time. By controlling the supply of nitrogen and phosphorus with respect to the RBCOD fed during the accumulation process, one can achieve an overall increase in the mass of PHA-rich biomass produced. The selected addition of nutrients to the accumulation process can be used to stimulate the non-PHA storing fraction of the biomass without decreasing the content of PHA in the biomass. A tendency for increase in non-PHA biomass production rates that exceed the PHA storage rate can be mitigated by supplying nutrients, such as nitrogen and phosphorus, at levels that are limiting with respect nutrient requirements generally understood for a process of biomass growth without PHA storage.

In one embodiment, an assessment is performed of the sources of nitrogen and phosphorus biologically available to the biomass for the PHA accumulation process. Biologically available nitrogen and phosphorus may be already present in the (harvested) mixed liquor containing biomass that is being disposed to the accumulation process. In this embodiment, the mixed liquor with biomass introduced to the accumulation process may need to be thickened prior to the accumulation process. If thickened, the excess water is removed, at least in part, in order to reduce the mixed liquor mass of nitrogen and phosphorus with respect to the mass of COD to be supplied for the accumulation process. After thickening and removing excess water from the biomass, the mixed liquor may be used directly. If necessary, the mixed liquor may be diluted with, for example, a dilution water containing negligible bioavailable COD, and negligible nitrogen and phosphorus. In this context, negligible nitrogen and phosphorus means of a sufficiently low initial nitrogen and phosphorus concentration such that, over the course of the accumulation process, the mess of biologically available nitrogen and phosphorus that the biomass is fed does not exceed the total mass of RBCOD to be consumed by the biomass in the relation of the limits of 15 mg-N/g-COD and 3 mg-P/g-COD.

Biologically available nitrogen end phosphorus may also be present in the influent stream or streams providing the supply of COD to be used by the biomass for the PHA accumulation process. In general, we find that the supply of nitrogen and phosphorus should be provided in step with the demand of COD to the biomass for sustaining a high respiration rate of the biomass in the accumulation process. In one embodiment, the influent containing COD supply for the accumulation process is pretreated, or blended with other supplies of COD or influents such that the supply of nitrogen and phosphorus that is fed to the biomass along with the supply of readily biologically available COD is kept within the range of 2 to 15 mg-N/g-RBCOD and 0.5 to 3 mg-P/g-RBCOD. Pretreatment or blending of influent streams are a means to either add, remove, or dilute the nitrogen and phosphorus concentrations, such that the RBCOD:N:P falls within the range to support, for a least some part of the accumulation process, a combination of PHA and non-PHA biomass production. Those skillful in the art will understand that other pretreatment processes, such as physical-chemical treatments, can also be used to remove nitrogen and phosphorus from water.

In the context of the current application, the biomass is divided into fractions of PHA-biomass and non-PHA biomass. The biomass is the sum of both those fractions. Generally, we consider for measurement purposes only the organic fraction of the biomass, that is to say the volatile organic solids (VSS) concentration of the reactor elements times the respective volumes. The “active biomass” in this context is the non-PHA fraction of the biomass, or, in other words, the total biomass (or the process VSS) minus the PHA-biomass. We say PHA-biomass because the PHA is stored as intracellular granules by the bacteria that comprise the biomass. The nutrient limits disclosed herein are designed to ensure with a greater degree of reliability that the mass increase rate in “active biomass” is equal to or less than the mass increase rate in PHA for the accumulation process.

In another embodiment, and where the accumulation process influent is with a COD:N:P within or below the range 2 to 15 mg-N/g-RBCOD and 0.5 to 3 mg-P/g-RBCOD, nitrogen and phosphorus may be added as influent streams that are separate from the main influent COD supply. In such a process, with a main influent of COD supply stream and separate influent nutrient supply, the influent flow rate of nutrients may be adjusted dynamically during the accumulation process. In this embodiment, biomass concentration and PHA content can be measured by relatively rapid off-line measurements or but also on-line using spectroscopy of light absorption including infrared or near infrared spectroscopy. One can consider in parallel both changes in suspended solids concentration (VSS) and PHA content of the suspended solids. From such measurements, the trend of PHA content of the biomass may be followed in time. The slope of this trend is important.
A positive slope indicates for conditions of PHA production rate greater than active biomass production rate. Under these conditions there is a not accumulation of PHA in the biomass as indicated by the overall increasing PHA content of the biomass.
A zero slope indicates for a balance between PHA production rate and active biomass production rate. Under these conditions, if the mass of biomass in the process is still increasing, it is indicated that the PHA-storing bacteria in the biomass are multiplying while the individual cells are, at the same time, storing PHA. It is generally known by experts in the field that when bacteria containing PHA divide, then both daughter cells inherit the same number of PHA granules. In other words, if a cell containing 6 PHA granules divides, then both daughter cells will contain 3 granules. The daughter cells both increase proportionally in “active mass” and PHA content before dividing again, growing and dividing again, and so forth. In this way the PHA content of individual cells is maintained with biomass growth resulting in a constant content of PHA in the overall biomass.
A negative slope indicates for PHA production rates that are less than active biomass production rates. In this case the biomass as a whole is increasing due to bacterial cell multiplication, or other non-PHA biomass production, but non-PHA biomass growth in general now out competes the biomass PHA storage activity.
   Without limitation, for example, the control strategy may comprise the following qualitative decision elements:
1. Positive Slope: PHA content increasing with:
   a. Decrease in specific COD demand, than increase nutrient supply rate; or
   b. Increase or constant specific COD demand, then keep nutrient supply rate constant or decrease nutrient supply rate.
2. Zero Slope: PHA content constant with:
   a. Decrease in specific COD demand, then increase nutrient supply rate; or
   b. Increase or constant specific COD demand, then keep nutrient supply rate constant or decrease nutrient supply rate.
3. Negative Slope: PHA content decreasing with:
   a. Decrease in specific COD demand, then terminate the fed-batch process; or
   b. Increase or constant specific COD demand, then decrease nutrient supply rate or terminate the fed batch process.

In keeping with the findings contained herein, the nutrient supply rate in such a control strategy should be maintained such that the COD:N:P supply is kept within the range of 2 to 15 mg-N/g-RBCOD and 0.5 to 3 mg-P/g-RBCOD.

The extent to which nutrients (nitrogen and phosphorus) can be used towards improving the productivity of PHA accumulation in biomass of mixed microbial cultures is poorly understood. The scientific literature concerning PHA production from mixed microbial cultures considers that PHA storage is best attained under the limitation of at least one nutrient in order to promote carbon substrate storage as PHA and reach a high level of PHA stored in the final biomass. Thus, the academic focus in this subject area has been on achieving as high a PHA content of the biomass as possible in a mixed culture PHA accumulation process. To the contrary, while a reasonably high PHA Content of the biomass is granted to be important, the focus of the present disclosure are methods and processes to achieve a generally improved productivity in PHA production from a mixed culture fed-batch PHA accumulation process. A high productivity of a fed-batch process for PHA accumulation relies not just on the overall content of PHA in the biomass; it is further dependent on the final total mass or amount of PHA produced per unit volume of reactor starting from an initial amount of biomass provided to the fed-batch process. The productivity may be further considered to be dependent on the PHA mass production rates per unit volume. Notwithstanding the different ways that productivity may be defined for a PHA production process, the present disclosure concerns the improvement of productivity for fed-batch PHA accumulation processes with respect to the amount of biomass provided to the accumulation process for every batch of biomass processed in the fed-batch accumulation. In other words, the methods and embodiments described herein are with the objective to produce a greater total mass of PHA within a reasonable range of PHA content in a fed-batch mixed culture accumulation process given:
1. An initial amount of biomass with anticipated PHA accumulation potential of greater than 40% (g-PHA/g-VSS) but with negligible initial PHA content.
2. A defined available time for achieving the PHA production from the supplied biomass that is generally longer than 12 hours but, due to practical considerations in costs and logistics of managing the stream of biomass from a BiPP, less than 72 hours.

Utilizing the methods disclosed herein, fed-batch processes that sustain a high biomass respiration rate under conditions of organic substrate “feed-on-demand supply” within a range of concurrent supply of nitrogen and phosphorus may support a combination and sustained balance of PHA and active biomass production. The effect of the combination of a restricted nutrient (N and P) supply in the feed, a restricted organic substrate availability (established by the maintenance zone environment), and high biomass respiration rate (established by the stimulation zone environment) results in a greater possible mass of PHA produced per mass of active biomass supplied to the process.

Too little supply of nitrogen and phosphorus constrains the fed-batch process to be one of exclusive or almost exclusive PHA production with little or negligible active biomass growth. In this case of nutrient starvation, the potential of the fed-batch process has not been exploited. If an optimal level of nutrients are provided with RBCOD, then a combination of PHA and active biomass production can result in at least a 25% increase and preferably more than at least a 50% increase in the total mass of PHA that could be produced from the biomass supplied to the accumulation process. Generally, greater process productivity can be gained by prolonging the period of biomass growth (with combined PHA storage) after the biomass has reached the PHA content expressed by its extant PAP.

Too high a supply of nitrogen and phosphorus, in combination with organic carbon supply rates well in excess of the biomass metabolic “demand”, increases the risk that non-PHA biomass production will dominate the process resulting in a net decrease in productivity. In this case of excess nutrient and organic substrate supplies, the potential of the fed-batch process and of the biomass for PHA productivity may have been lost because biomass is produced without PHA.

The embodiment of delivery of carbon substrate on demand in a process that sustains an overall high biomass respiration rate for purposes of PHA storage with high molecular weight has been previously disclosed in WO 2011/070544A2. Processes of fed-batch feed-on-demand are described in which a biomass respiration capacity is sustained by repeated stimulation of fractions of the biomass In at least one stimulation zone. However, conditions of nutrient supply that tend to promote, during at least some part of the fed-batch process, a combination of growth and PHA storage in the biomass have not been previously disclosed.

A biomass comprising a mixed-culture with an enriched PHA accumulation potential may be expected to accumulate a significant amount of PHA to levels in excess of 0.40 g-PHA/g-VSS. More frequently in practice, mixed culture PHA accumulation results of well in excess of 0.50 g-PHA/g-VSS have been demonstrated. These high and even extreme levels of PHA accumulation potential in a mixed culture biomass are often demonstrated by feeding just RBCOD to the biomass in absence of supplying nitrogen and/or phosphorus to the biomass (see, for example, open culture results of Johnson et. al., Biomacromolecules 2009, 10, 670-676). However, the recipe for adding nutrients during such an accumulation process in order to stimulate a combination of active biomass production along with PHA accumulation and without sacrificing the PHA content of the biomass has not been previously described.

We found that for a biomass with a PHA accumulation potential of between 40 and 70 percent (g-PHA/g-VSS), a COD:N:P ratio of the feed should lie between 200:0.4:0.1 and 200:3:0.6. By adding restricted amounts of bio-available nitrogen and phosphorus along with RBCOD, and with a feed-on-demand process control strategy, we found an increase in productivity of PHA from the same initial source of active biomass. This increase in productivity was with respect to accumulation of PHA from the same source of biomass, and with the same RBCOD, but without adding nitrogen or phosphorus to the feed. When excess nutrients were added (i.e. with COD:N:P in excess of 200:5:1) with the same supply of RBCOD, we observed an increase in risk that the biomass would grow in active biomass without reaching its anticipated potential in PHA accumulation potential.

The present disclosure relates to methods directed towards a fed-batch PHA production process for mixed-cultures under open culture process conditions, whereby improved process productivity is achieved by ensuring:
High biomass respiration with restricted “on-demand” availability of a readily biodegradable carbon source, in combination with
Restricted availability of both nitrogen and phosphorus,
   so as to promote restricted active biomass growth during the fed-batch process. Sustaining a combination of growth and PHA storage in the biomass can be readily monitored on-line and/or off-line during the course of the accumulation process. The fed-batch process can be terminated at a point when time or productivity goals have been achieved (criteria of diminishing returns), or when conditions of growth overtake PHA storage such that an onset of decrease in productivity is detected. Such a decrease in productivity may be due to, for example, an onset of growth in excess of the PHA storage rate leading to a progressive decrease in the PHA content of the biomass.

Microbial biomass in a mixed microbial culture producing PHA comprises organisms being able to accumulate PHA as an intracellular granule and those organisms that do not store PHA. Again, active biomass herein designates the amount of total biomass less the PHA mass of the sample. One objective of the methods and processes disclosed herein is to create conditions during the fed-batch process so as to promote PHA accumulation in tandem with the preferential growth of that fraction of the active biomass that grow while they continue to store PHA.

Reaching an increased mass of PHA produced per unit volume within a reasonable time for the fed-batch accumulation process necessitates promoting a combination of active biomass growth and PHA storage, specifically with respect to growth and storage rates per unit volume. Such a combination can be influenced by the amount of nutrients with which the biomass is fed in relation to readily biodegradable organic carbon or RBCOD as the principal substrate. A nutrient addition is selected such that the PHA content in the biomass is at least constant or, more preferably, increasing in time. Typically used RBCOD for PHA storage are volatile fatty acids. Herein, we have discovered conditions for driving fed-batch PHA accumulation processes necessary to achieve increased biopolymer productivities in a mixed microbial culture wherein the strategy is to stimulate both growth of PHA-storing active biomass with PHA storage such that the PHA content of the biomass is sustained at a significant level of greater than 40% g-PHA/g-VSS for an extended period of time (6 hours to 72 hours). The challenge in mixed microbial cultures is to encourage preferentially the growth of such PHA-storing organisms during the accumulation process. We have observed that the preferential growth of PHA-storing organisms in the biomass and PHA storage activity can be achieved in a fed-batch system under the combined conditions of:
1. Co-feeding nutrients nitrogen and phosphorus with RBCOD within a range of selected N/COD (as mg-N/g-RBCOD) and P/COD (as mg-P/g-RBCOD) ratios,
2. Supplying COD in such a way so as to sustain maximum or near maximum respiration rates, and
3. Maintaining the biomass otherwise in an environment of minimal availability of COD so as to limit carbon substrate to be “on-demand” and to thereby impede an onset of a kind of active biomass growth that is not concurrent PHA storage.

Many waste streams in need of bio-treatment contain nutrients in addition to readily biodegradable COD; therefore, efficient PHA production from such streams relies on establishing the practical working range of COD:N:P yielding the highest PHA productivities. In cases involving COD streams richer or poorer in N or P than necessary, a target COD:N:P in the feed can be tailored by combining other sources of COD or N and P or removing N and P as necessary in order to arrive at COD:N:P ratios providing for the goal of sustaining PHA storage with PHA-storing active biomass growth. We have found that such practice can be applied towards significantly improving the overall productivity in such fed-batch processes.

### EXAMPLE 1

### Evaluation of the Effects of N/COD and P/COD Feed Supply Ratios in a PHA Production Process Using Either Acetate or an Industrial Process Water Containing a Mixture of VFAs as RBCOD

Two series of feed-on-demand fed-batch PHA production experiments were performed with two respective RBCOD sources. Each series of experiments comprised the evaluation of the PHA production fed-batch runs over a range of applied N/COD (mg-N/g-RBCOD) and P/COD (mg-P/g-RBCOD) ratios in the process feed. For all experiments the same source of supply of biomass was used.

### Materials and Methods

The source biomass was a mixed culture of activated sludge that was enriched with a significant PHA accumulation potential. This biomass was produced in a pilot-scale SBR (400 L). The pilot-scale SBR (Biomass Production Process or BiPP) had been operated for more than 3 years under aerobic feast-famine selection conditions at an OLR=1-1.6 g-COD/L/d, SRT=4-8 d, and HRT=1-2 d with fermented cheese whey permeate as the source of RBCOD for the feed. The surplus biomass harvested from the end of the SBR cycle (famine) was with a consistent performance in PHA storage capacity. Thus, this biomass was used as a source of enrichment biomass with which to test for the influence of nutrient levels with a given RBCOD on PHA production productivity.

The accumulation fed-batch set-up consisted of two parallel operated, 1-L reactors (0.5 L operating volume), each equipped with DO (Oxymax W COS41, Endress+Hauser), pH (H63 Schott Instruments, Reagecon standards; Liquisys M CPM223/253 transmitter), and temperature probes, a diaphragm feedstock-dosing pump (Grundfos DME), a magnetic stirring plate (400 rpm), an air pump for air supply, and a temperature regulated water jacket. For each accumulation experiment, the two fed-batch reactors were operated in parallel. One fed-batch reactor was a reference using always the same N/COD and P/COD ratio, and thereby served as an experimental control to confirm the consistency of the biomass performance over the course of the respective experimental series. In the second fed-batch reactor, a range of different N/COD or P/COD ratios were applied. Substrate delivery was semi-continuous in triggered pulses based on a feed-on-demand respirometric control (WO 2011/070544 A2). Note that in such a small-scale laboratory process, it is practical to combine the stimulation and maintenance volumes, and thereby stimulate all the biomass in the process synchronously. At industrial scale, the methods embodied by WO 2011/070544 A2 disclose a means of an asynchronous stimulation of the biomass, whereby fractions of the biomass are stimulated at a given time in a separate stimulation zone.

The accumulations were run for 24 hours at a constant temperature of 30° C., with dissolved oxygen levels greater than 2 mg/L, and substrate stimulating substrate concentrations per pulse of 200 mg-RBCOD/L. Grab samples of the process mixed liquor from both reactors (4-10 mL) were collected at selected time points during the accumulation for monitoring the process performance.

Two series of accumulation experiments were performed with two different RBCOD sources: fermented cheese whey permeate (FWP) and acetate. FWP (Table 1) was produced in a pilot-scale CSTR (V=200 L) operated with an HRT of 8-12 d and the fermented effluent was stored refrigerated at 4° C. before use. The FWP was the same RBCOD source that was used as the feedstock used for the biomass production in the 400-L pilot BiPP.

In an initial set of four accumulation experiments, the objective was to evaluate the effect of the N/COD ratio on the biomass response. Here, the reference reactor was fed the FWP control feed that contained a defined level of N/COD and P/COD (Table 2). The parallel reactor was fed the same FWP with supplements of NH₄Cl so as to cover a selected range of feedstock N/COD ratios (Table 2). In one of the accumulations, N and P starvation conditions (N/COD=0 and P/COD=0) were applied by using a VFA mixture mimicking the VFA composition and pH of the FWP (Table 2). Phosphorus was considered to be in excess in the FWP (Table 1) and, therefore, in this first set of experiments only N/COD ratios were varied systematically. Before each experiment with FWP, the SBR biomass was centrifuged (4000×g, 5 min) and then resuspended in tap water in order to remove the potential for influence of experimental variation due to changes of residual nutrient levels in the biomass mixed liquor sourced from the BiPP.

The second set of seven accumulations was conducted with a feedstock COD of sodium acetate (50 g-COD/L) and a range of NH₄Cl and KH₂PO₄ levels was applied to target different N/COD and P/COD ratios in the feed (Table 3). In these accumulation experiments, the SBR biomass was centrifuged (4000×g, 5 min) and re-suspended in a buffer solution (0.248 g-Na₂CO₃/L, 0.262 g-NaHCO₃/L, 0.5 g-MgSO₄.7H₂O/L, 0.25 g-CaCl₂.2H₂O/L) so as to maintain a consistent matrix with which to compare the influence of nutrient levels In the feedstock. The pH was between 7-8 during the accumulations. Micronutrients were provided by a single addition of 0.2 and 0.6 mL of an Fe/Zn stock solution and a trace element solution, respectively, at the start of the experiments. The Fe/Zn solution contained 7.8 g-FeCl₃.6H₂O/L and 0.78 g-ZnSO₄.7H₂O/L, and the trace elements solution contained 0.25 g-H₃BO₃/L, 0.25 g-CoCl₂.6H₂O/L, 0.205 g-MnCl₂.2H₂O/L. 0.1 g-NaMoO₄.2H₂O/L, 0.05 g-CuSO₄.5H₂O/L, and 0.3 g-Kl/L. The pH of the feed was adjusted to 3.5 with 4M NaOH. A fixed COD:N:P ratio in the feed of 100:1:0.9 was applied as the experimental control in the reference reactor. In the parallel accumulation reactor, the applied COD:N:P ratios covered N and P conditions of starvation, limitation, and excess. The definitions of limitation and excess were defined based on observed nutrient removal by the same SBR biomass in the first set of accumulations with FWP (Table 2). In both sets of experiments, the reference accumulations were used to evaluate the reproducibility in performance of the SBR biomass in PHA accumulation potential over the periods in which the experiments were performed.

Mixed liquor samples from accumulation reactors were centrifuged (4000×g for 5 min) and the centrate was then filtered (Munktell MGA, 1.6 μm). The filtrate was analyzed by Hach-Lange methods (LT100, Xion 500 spectrophotometer), using distilled water dilutions as necessary, for soluble COD (LCK114), N—NH₄⁺ (LCK 138 and 238) and P—PO₄³⁻ (LCK 349 and 350). VFA concentrations were measured by gas chromatography (Perkin-Elmer Autosystems) after further filtration through a 0.22 μm filter as per Morgan-Sagastume at al. (2010. Water Res. 44:5196-5211) with minor modifications; an internal standard, containing 25% formic acid and 3 g L-1 crotonic acid, was added to the 0.9 mL of filtered sample.

The biomass pellets were dried at 70° C. overnight, weighed for TSS measurements and then used for PHA analysis. Mixed liquor TSS and VSS were measured at the start, middle, and end of the accumulations based on standard methods (APHA, 1998). The biomass pellet PHA content was measured as previously reported elsewhere (Werker at al. 2008. Water Res. 42:2517-2520. P(3HB) and Glucose (Aldrich 36,350-2) were used for calibration standards for 3HB and polysaccharides, respectively.

The PHA content in the biomass was calculated as g-PHA per g-VSS. Yields were calculated on a COD basis by converting the amount of mass PHA formed (1.67 g-COD/g-PHB and 1.92 g-COD/g-PHV) by the consumed COD. Active biomass (Xₐ) was defined as VSS less PHA content. For considering biomass production on a COD basis, a conversion factor of 1.42 g-COD/g-Xₐ was used to represent the active biomass on a COD basis assuming a nominal biomass composition of C₅H₇NO₂. The overall biomass yield was therefore the estimated VSS produced on a COD basis with respect to the consumed RBCOD.

Empirical asymptotic or quadratic functions were used represent the mass balance trends in time (t) from the measured values of biomass PHA content, VSS production and COD consumption. The derivative of the respective time-based empirical functions was used to estimate the trends for accumulation parameter rates of change.

### Results and Discussions

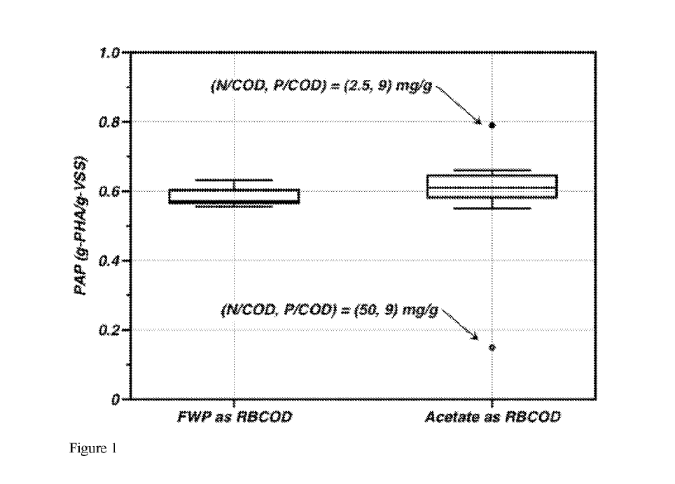

FIG. 1 indicates the consistency of the performance of the biomass in expressed PHA accumulation potential over 22 PHA production experiments spanning a number of months. Generally, the biomass exhibited a PAP of between 50% and 70% (g-PHA/g-VSS). Outliers in the PAP were observed for the acetate RBCOD and were associated with a nitrogen limitation and nitrogen excess with phosphorus otherwise in balance. The outcome of N in excess with P in balance suggested that performance with excess nutrients may not be sufficiently robust. In any event, it is of no advantage to add nutrients in excess for an accumulation process due to costs of nutrient additions (if required) and/or due to costs of the PHA production effluent management. Ideally, the effluent from the PHA production process should be, as best as possible, a treated wastewater in order to avoid unnecessary operational costs and investments in the process effluent management.

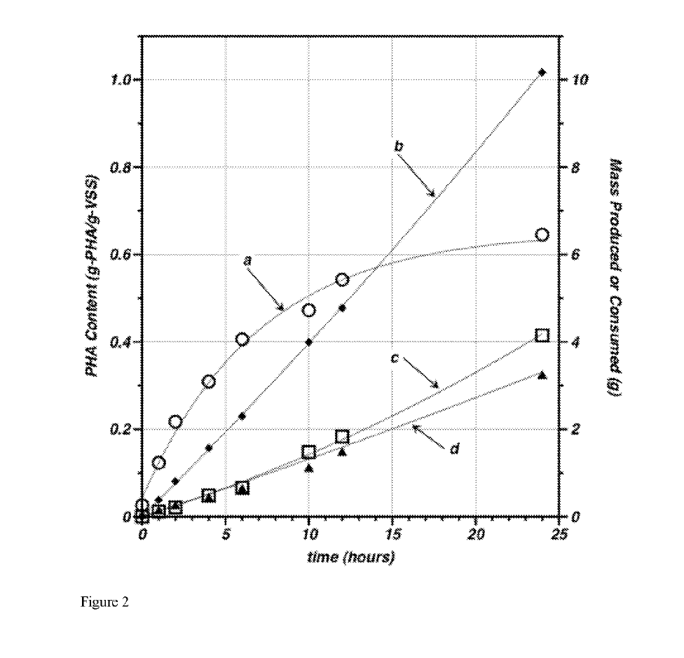

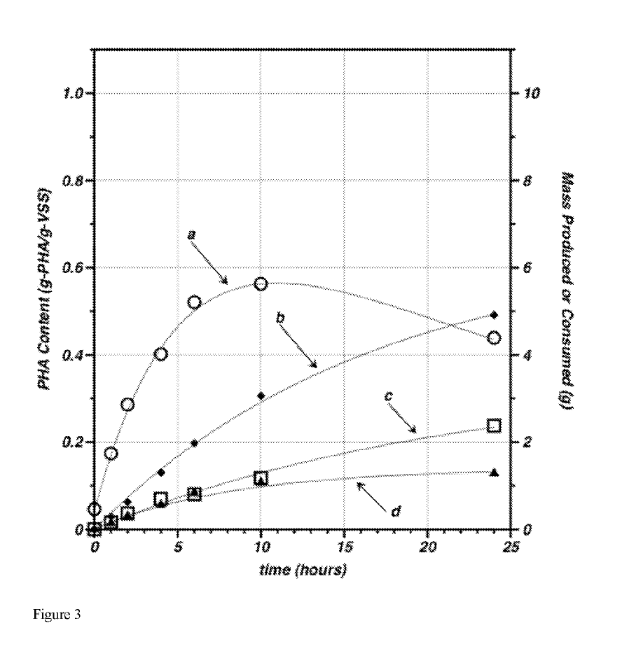

FIGS. 2 and 3 illustrate the typically observed trends of PHA accumulation for the acetate and FWP RBCOD substrates, supplied by feed-on-demand process methods in all cases. In these figures typical empirically fit trends, of biomass PHA content (a), RBCOD consumption (b), biomass (VSS) production (c), and PHA production (d), are shown. In FIG. 2, an onset of active biomass growth is observed by the trend of biomass production exceeding PHA production over time. All the while, the PHA storage rate is greater than or equal to the active biomass production as shown by the slope of the PHA content that is always greater than or equal to zero. FIG. 3 shows how, for the case of an FWP reference accumulation, the active biomass production began to take over the PHA production activity due to an observed maximum in the PHA content in time, and an eventual negative slope in the trend of PHA content was observed.

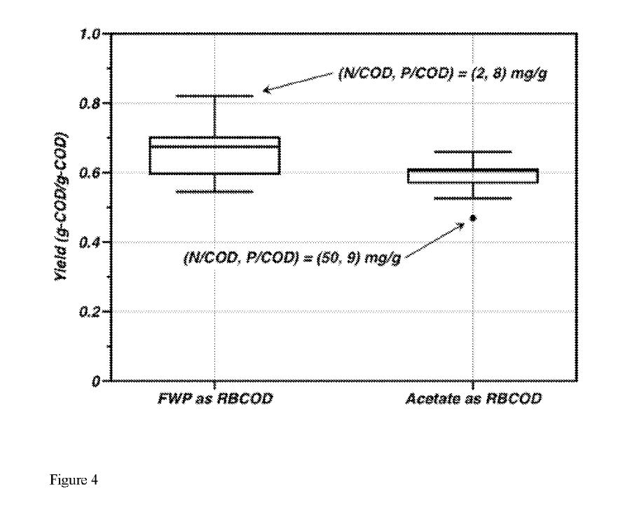

The overall yield of biomass on RBCOD was relatively consistent (FIG. 4) although the yield of biomass on RBCOD was on average greater for the case of the FWP as RBCOD source. The biomass was sourced from a process treating FWP, therefore one may expect a greater level of acclimation to the more diverse organic content of this feedstock. Once again, outliers were associated with limited (FWP upper whisker) or excess (Acetate outlier) nitrogen supply. Thus, the poorest yield of biomass on RBCOD was for the case of excess nitrogen. This observation was not reproduced for the excess nitrogen using FWP as RBCOD. Given that all factors are difficult to control for in biological systems, we considered the conditions of excess nutrients to introduce the most risk for the industrialized process reliability due to lack in a stringent constraint to restrict non-PHA biomass growth based on partial limitation of nutrients. However, it was nevertheless of practical significance to observe for the case of FWP accumulations that a good productivity in PHA production can in some cases be successfully achieved even for RBCOD supplied with excess nutrients.

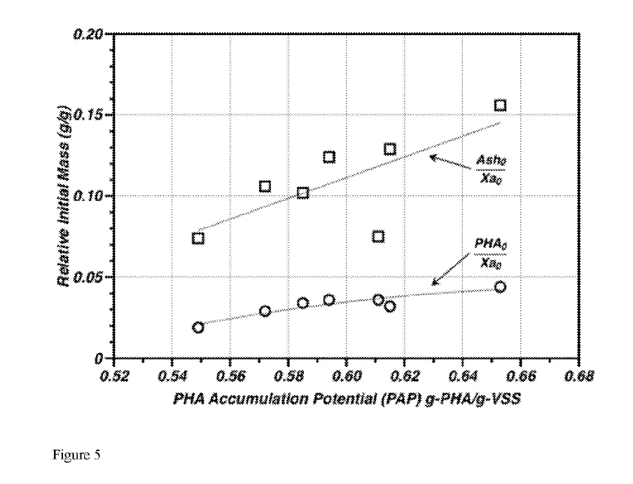

It was important to observe (FIG. 5) that even with all things being equal for the PHA accumulation process, some variability in PAP may nevertheless be expressed by biomass coming from the same source. An observed correlation was noted with respect to the expressed maximum PAP of the biomass with acetate as RBCOD and for the replicated control (reference) conditions of applied nutrient balance ((N/COD, P/COD)=(10.9) mg/g). Hare, Ash₀ refers to the initial biomass inorganic mass, PHA₀ refers to the initial biomass PHA, and Xa₀, refers to the initial active biomass. Differences in initial PHA and inorganic material contents of the biomass suggested more or less “starvation” stress of the biomass used for the 7 sets of experiments in the series with acetate as RBCOD. Therefore, notwithstanding these observations and findings of methods and process to optimize the productivity in PHA production for mixed cultures, the biomass history from the source can also introduce some variability in the outcomes of productivity in the PHA production process.

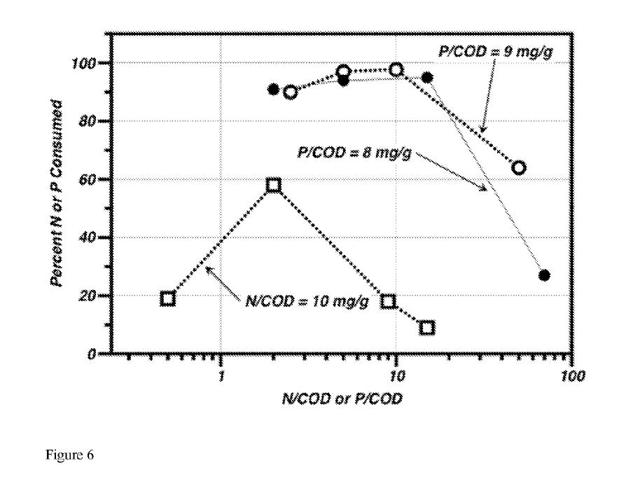

The principal objective of the investigation was to establish the optimal nutrient range for mixed culture PHA production given a biomass expressing a PAP of between 40 and 70 g-PHA/g-VSS. Sufficient nutrients should be provided in order to support active biomass production required to achieve improved process productivity. However, excess nutrient addition was to be avoided due to a loss in process constraint that would restrict undue non-PHA storing biomass growth, and due to a necessary base requirement to maintain the best possible process effluent water quality standards. In FIG. 6, data of nutrient consumption from the Acetate RBCOD series (open symbols) and from the FWP RBCOD series (closed symbols) are presented. Square symbols are for percent P consumed as a function of P/COD applied and for constant applied N/COD as noted. Circular symbols are for percent N consumed as a function of N/COD applied and for constant applied P/COD as noted.

The data of the percent consumption of applied nutrients, summarized in FIG. 6, suggested an optimal P/COD of approximately 2 mg-P/g-COD with N/COD in limitation (10 mg-N/g-COD). For the cases of P/COD in excess, these data indicated that N/COD beyond approximately 15 mg-N/g-COD was in excess.

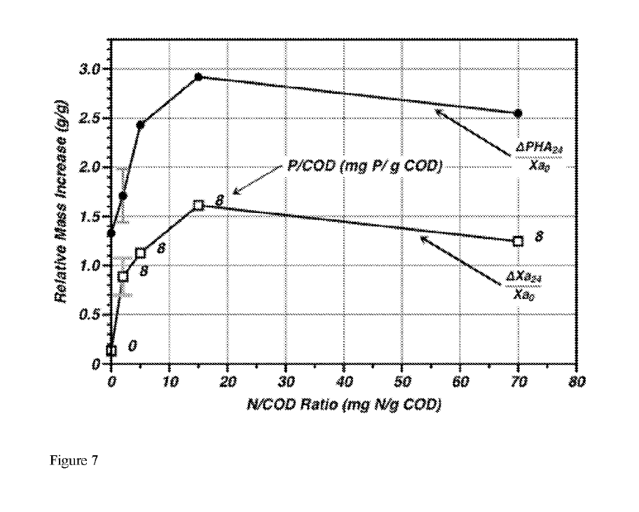

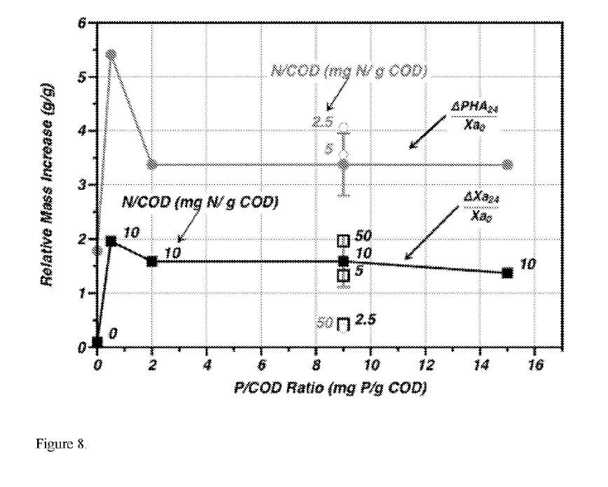

If nutrients are supplied at levels that are limiting or near-limiting (as shown from the data in FIG. 6), then we found (FIGS. 7 and 8) that a fed-batch PHA production process can be reliably operated based on the feed-on-demand strategy with a significant increased productivity in the mass of PHA produced per mass of active biomass supplied to the process. If nutrient starvation is applied, the biomass may still express a similar PAP, but in absence of any active biomass growth, the productivity of PHA will be less. Under nutrient starvation, there is little or no opportunity to extend the operation time in order to gain more PHA production because the biomass becomes increasingly more unresponsive in respiration once the PAP is expressed. In this case, added RBCOD generally begins to accumulate in the mixed liquor. If nutrient excess is applied, then we observed a risk that the onset of active biomass growth without PHA storage is no longer constrained. In this case of nutrient excess, the expression of a low PAP and/or an onset of active biomass growth rate in excess of PHA production rate limit the length of time the PHA production process can be driven. Constrained supply of nutrients nurture conditions to achieve a prolonged period of accumulation where the PHA content of the biomass remains relatively constant, but the mass of material produced progressively increases in time. In this case the process productivity increases the longer the process is kept in operation. The length of operation may in this case become restricted in industrial practice due to the supply rate of biomass from the BiPP and the ongoing need to manage this, now, value added stream with the available resources of tanks, and RBCOD supply for the PHA production process.

### EXAMPLE 2

### Demonstration of a Preferred Embodiment at Pilot Scale

The potential for increased PHA production by feed-on-demand in a fed-batch accumulation process was demonstrated at pilot scale. Selected N/COD and P/COD substrate ratios were applied using acetate as RBCOD for PHA production from a waste activated sludge. The biomass was enriched in PHA accumulation capacity in the course of biological treatment of a municipal wastewater.

### Materials and Methods

Two fed-batch accumulations were conducted with feed on demand controlled based on respirometry (WO 2011/070544A2), with and without N and P addition, using acetate as the RBCOD source (83-100 g-COD/L feed stock, pH adjusted to 5 with NaOH). In the accumulation with nutrient addition, NH₄Cl and KH₂PO₄ were added to the feed with a COD:N:P ratio of 100:1.2:0.07 (mg-N/g-RBCOD=12, mg-P/g-RBCOD=0.7). The target N/COD and P/COD values were within the range determined in Example 1 to yield increased PHA productivities, and the target respiration stimulating concentration in the reactor ranged between 80 and 110 mg-COD/L.

A 400 L accumulation reactor with an initial activated sludge biomass level of approximately 1 g-VSS/L was used. The activated sludge was sourced from a pilot scale reactor treating a municipal wastewater under established conditions of aerobic feast-famine selection for increased PHA accumulation potential (WO 2012/023114 A1). Water quality analyses from grab samples taken during me PHA production process were performed similarly as described in Example 1.

Both accumulations were conducted at 25° C. and lasted 20 h. The initial levels of soluble total N in the two experiments were not exactly the same. Without N and P addition the N concentration was initially 13 mg-N/L, and with N and P addition the starting concentration was 6 mg-N/L.

### Results and Discussion

The available N (initially present and/or added) in the tests was assimilated by the biomass over the course of the respective accumulations (removal efficiencies≧92%). The results of this demonstration of principle at pilot scale are summarised in Table 4, Nutrients supplied with the range of the preferred embodiment and applying feed-on-demand methods of PHA accumulation resulted in an 85 percent increase in the specific PHA production over the 20-hour production process with respect to the reference conditions of P-starvation.

### EXAMPLE 3

### Embodiments for Commercial Implementation

Without limitation, a practical representation of implementation of the findings is provided schematically in FIG. 7. With reference to FIG. 7, a wastewater treatment process (2) receives influents (1) and improves the water quality in order to meet the requisite standards for the effluent water quality (3). A biomass is produced in the process, and the biomass is separated from the process (4) whereby excess water from the mixed liquor is discharged (5) with similar constraints on the effluent water quality. Dilution water (6) may be admixed to the dewatered biomass, and the biomass harvested from (2) is disposed to a fed-batch PHA accumulation process (7).

The biomass from the water treatment facility is enriched with a significant potential in capacity for PHA accumulation. Examples of embodiments teaching in the art of enrichment may be found in US 2010/0200498, WO 2011/070544A2, WO 2011/073744A1, WO 2012/022998A1, and WO 2012/023114A1. This enrichment results in allowing the biomass disposed from (2) to (7) to accumulate PHA with the result of a PHA-rich-biomass (8) containing at least 40%, and preferably more than 50%, of its dry weight (g-PHA/g-VSS) as PHA. Examples of process embodiments of (7) may be found in WO 2012/022998A1. The PHA-rich biomass (8) may be further processed to ensure thermal stability of the PHA-in-biomass as taught by embodiments in WO 2012/022998A1. The effluent (9) from the PHA accumulation process (7) must similarly meet, or be made to meet, as for effluents (3) and (5), water quality standards for discharge.

There may be any one of a number influent streams (12A, 12B, 12C, 12D, etc.) supplying COD and/or nutrients (N and P) to the accumulation process (7). Some or all of the influent streams may need some form of pretreatment (13 and 14) as means to either improve the quality of the RBCOD content of the feed or else to adjust (increase or decrease) the nutrient content of the source. The influent streams can be blended as necessary (15) in order to arrive at a supply of substrate of RBCOD to (7), such that, on average, nutrients are supplied with RBCOD in the range of 2 to 15 mg-N/g-RBCOD and 0.5 to 3 mg-P/g-RBCOD. Some embodiments may further include one or more methods and or devices to measure the process water quality, biomass production, and biomass PHA content and thereby provide feedback for control strategies to adjust the blend and rate of supply of influent streams and/or the overall process. For example, the accumulation process includes off-line and/or on-line measurements, which provide trends in time that reflect the development of the biomass respiration, biomass concentration, and biomass PHA content. Based in this information a process control response (11) to the process monitoring (10) is used to establish the feed-on-demand RBCOD loading rate for the process and to adjust the nutrient balance in the feed within the range of 2 to 15 mg-N/g-RBCOD and 0.5 to 3 mg-P/g-RBCOD.

### Example 4

### Stimulation and Maintenance of Biomass Respiration

By way of a simple laboratory exercise, the principle of respiration stimulation was demonstrated in a practical experiment. Without limitation, activated sludge from a pilot scale biological nitrogen removal process treating a municipal wastewater was employed. The biomass was enriched to exhibit a significant PHA accumulation potential based on a feast-famine strategy of selection using anoxic-feast with aerobic famine, as has been recently demonstrated (Anterrieu at al. 2013. New Biotechnology, DOI—10.1016/j.nbt.2013.11.008). Activated sludge was harvested from the pilot process at the end of an aerobic famine cycle,

In a non-aerated 100 mL vessel, the biomass at a concentration of 5 g-VSS/L was dosed with acetate to reach a respiration stimulating RBCOD concentration of 100 mg-COD/L. The dissolved oxygen of the vessel was negligible and the contents were mixed for 1 minute representing a time of respiration stimulation.

After this period of stimulation, the biomass was transferred to a well-mixed maintenance reactor containing no biomass but a dilution water volume of 700 mL. The dilution water was pre-saturated with dissolved oxygen (DO), and the biomass respiration rate was thereby evaluated from the linear trend in time of dissolved oxygen decrease. Aeration to the dilution vessel was introduced once the dissolved oxygen decreased to about 5 mg-O₂/L. The dissolved oxygen generally would increase to a steady-state value less than the saturation value. A sudden subsequent increase in dissolved oxygen indicated for the full consumption of the added RBCOD. Even though the RBCOD was diluted by 8 times, the respiration level that was established by the stimulation zone contact concentration was maintained after the substrate dilution to approximately 12 mg-COD/L.

After the substrate was consumed and the dissolved oxygen increased again to a steady state DO concentration that was near the initial saturation value, a second pulse of acetate was added to the biomass. This time, the same mass of COD was added, but this mass was added to the biomass that was now in the maintenance reactor. Just prior to adding this second aliquot of substrate, the aeration in the maintenance reactor was turned off and so the respiration rate could be similarly monitored as well as the period of substrate consumption as before. By adding the same mass of substrate to the maintenance zone, a much lower “stimulating concentration” was established, nominally 12 mg/L.

In this example, the non-aerobic respiration stimulation at 100 mg-COD/L resulted in a respiration rate of 0.24 mg-O₂/L/min. By adding the same mass of substrate without the benefit of the contact time at higher concentration in the stimulation reactor, the respiration for the same biomass was only 0.18 mg-O₂/L/min. Thus, a 30% higher respiration rate was established in the biomass by the use of a stimulating reactor, and this higher respiration rate could be maintained in an environment of significantly lower RBCOD concentration within a maintenance zone or reactor.

In a second set of simple experiments, the respiration of a biomass with a nominal PHA accumulation potential of between 50 and 60 percent (g-PHA/g-VSS) was stimulated from a level of an endogenous respiration rate to a level of a feast respiration rate by an impulse addition of acetate to reach a stimulating RBCOD concentration of 200 mg-COD/L. The biomass stimulating vessel contained 1 litre mixed liquor that was well-mixed with a VSS concentration of about 1800 mg-VSS/L. A small aquarium pump connected to a stone diffuser in the vessel was used for aeration.

An experiment proceeded as follows: the dissolved oxygen was brought up to between 7 and 8 mg-O₂/L after which time the aeration was turned off, the dissolved oxygen concentration was monitored and DO values were logged every 10 seconds. At a selected point in time once the dissolved oxygen had dropped to below 6 due to the biomass endogenous respiration, the acetate was pulsed into the reactor and the respiration response was monitored by an onset of an increase in rate of dissolved oxygen consumption. Once the dissolved oxygen dropped below 2 mg-O₂/L, the aeration was turned on and the dissolved oxygen level was followed until the levels increased once again to a steady state value between 7 and 8 mg-O₂/L indicating substrate consumption and a return to an endogenous level of respiration. The procedure of respiration stimulation was then repeated two times and the whole experimental procedure was duplicated.

The trends in dissolved oxygen were corrected for the empirically determined first order delay constant for the dissolved oxygen probe, and the trends in slope from endogenous to stimulated respiration rates were fit by least squares regression analysis. From these data, the time of delay, from the point in time of impulse addition of substrate to the vessel, to the point in time indicative of a stimulated respiration, was estimated. The response time for biomass stimulation of increased respiration due to a sudden increase in substrate concentration was relatively short and, without consideration of the mixing time scale necessary to reach a uniform substrate concentration in the vessel, it was estimated to be 12±3 seconds. Therefore, a minimum time necessary, for biomass to be influenced by a higher RBCOD concentration in the stimulation zone in a preferred embodiment of the accumulation process, was conservatively considered to be of a time scale order of magnitude of 1 minute.

### Control System Embodiments.

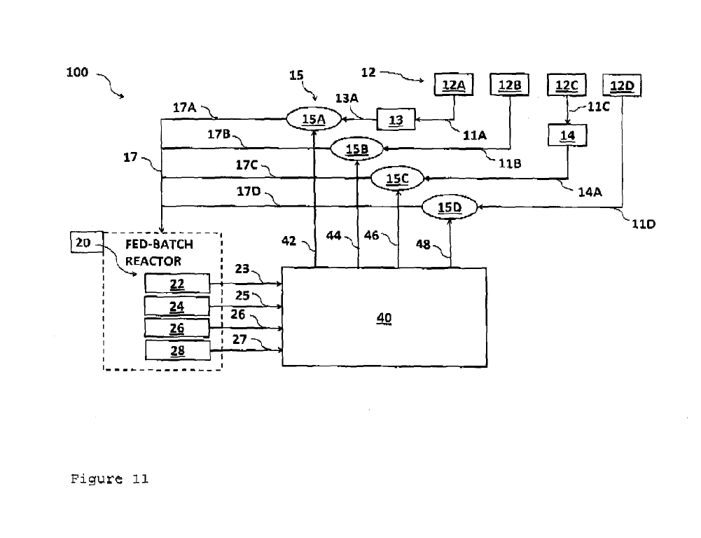

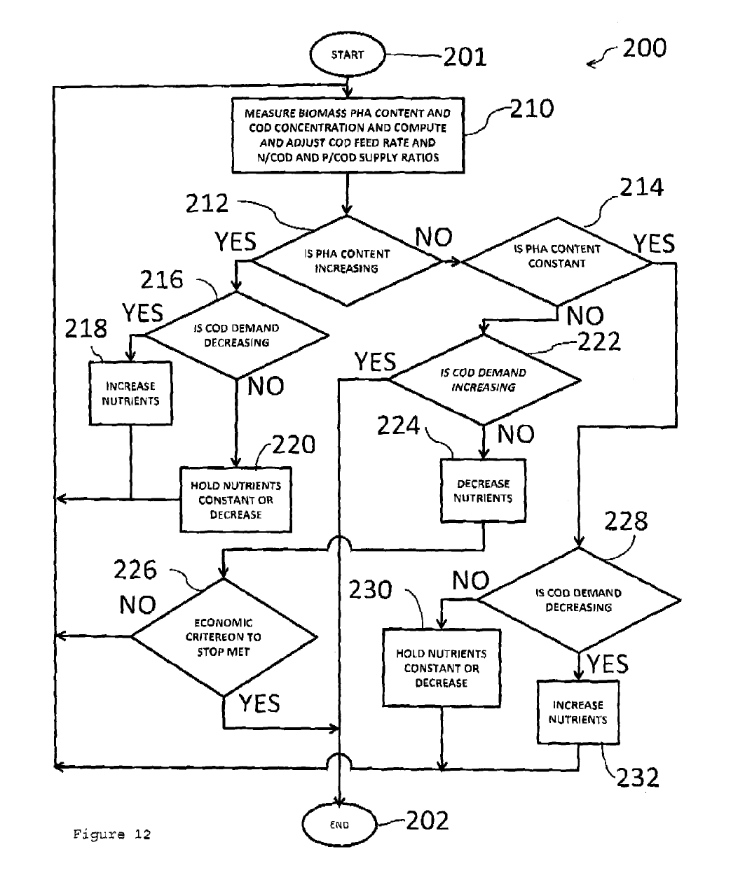

As discussed above, the method or process described herein entails maintaining ratios of phosphorus and nitrogen to RBCOD within a range. In one embodiment, on average nitrogen to RBCOD ratio is generally maintained within a range of 2 to 15 mg-N/g-RBCOD while on average the phosphorus to RBCOD ratio is maintained within the range of 0.5 to 3 mg-P/g-RBCOD. FIG. 11 shows a control system 100 that is designed to control the makeup (especially nitrogen, phosphorus and RBCOD) of a feed stream that is directed to one or more tanks of a fed-batch reactor and forms a part of a PHA accumulation system. The control system shown in FIG. 11 is one exemplary control system. There are others. In addition, FIG. 12 is a logic control diagram that describes one exemplary logic control for controlling the blending of nitrogen, phosphorus and RBCOD into a fed-stream being directed to the fed-batch reactor. As with the control system 100, the control logic diagram 200 of FIG. 12 is but one exemplary embodiment of such a logic control approach.

Turning to FIG. 11, the control system 100 is shown therein. Control system 100 is designed to control amounts of RBCOD, N and P directed into a PHA accumulation process. As described above, the method of the present invention entails controlling the ratios of nitrogen and phosphorus to RBCOD within a range enhances the mass of production of PHA from the biomass supplied to the accumulation process.

Control system 100 includes a series of sensors indicated generally by the numeral 20. The sensors embody measurements that may be conducted both off-line and on-line to the process but generally the data from the sensing is provided within a timeframe that is short relative to the timeframe of the accumulation process (typically minutes or tens of minutes). The sensors 20 are adapted to sense levels of process variables in a fed batch reactor that forms a part of the PHA accumulation system. In addition, control system 20 includes a controller 40 for receiving data input from the sensors 20 and determining control actions. Associated with the controller 40 is a series of injectors 15 that are operatively connected to a series of nutrient sources 12. In one embodiment, the nutrient sources 12 include one or more sources for phosphorus, one or more sources for nitrogen and one or more sources for RBCOD. There are at least two independent sources to the process whereby each source is distinct in at least one manner with respect to concentration of RBCOD, N, or P.

Sensors 20 include sensor 22 for sensing the concentration of the biomass and the level of PHA in the biomass disposed in the fed batch reactor of the PHA accumulation system. Sensor 24 is employed for sensing the COD or RBCOD level of the mixed liquor in the fed batch reactor. Sensor 26 functions to sense the nitrogen level in the mixed liquor and sensor 28 functions to sense the phosphorus level in the mixed liquor. Sensors 20 are capable of generally continuously monitoring these process variables. In one embodiment, one or more of the sensors may incorporate spectroscopy, such as infrared sensing. Signals from the sensors 22, 24, 26 and 28 are directed by way of conductors 23, 25, 27 and 29, respectively, to the controller 40. Although this exemplar embodiment shows measurements occurring in the reactor, one of skill in the art appreciates that embodiments or elaborations of this illustrative example exist where other variables such as concentration may also be measured in the feed and used as an input to the controller logic.

Controller 40 includes input signal conditional capability known to those of ordinary skill in the art. Further, the controller is operative to implement logic to form commands to be communicated to the injectors 15 for the purpose of controlling the ratios of N to RBCOD and P to RBCOD. Controller 40 also implements logic for determining when the processes occurring in the fed batch reactor have created conditions that call for harvesting PHA-rich biomass, and otherwise ending or suspending the processes. Generally, these conditions are based on the rate of production of PHA in the biomass with respect to the rate of biomass production in general, and consideration of incremental cost and benefits and/or practical limitations for continuing the PHA accumulation process in the fed batch reactor.

Turning now to portions of the system comprising injectors 15 and nutrient sources 12, it is appreciated that each nutrient source is in fluid communication with a corresponding injector 15. Nutrient sources 12 can include various nutrient sources. In one preferred embodiment, the nutrient sources 12 include in combination a source of RBCOD, nitrogen and phosphorus. Sources 12 may also include mixtures containing RBCOD, nitrogen and phosphorus. If necessary, nitrogen, for example, may be provided as an independent source as ammonium chloride (NH₄Cl), for example, and phosphorus may be another independent source as potassium phosphate (KH₂PO₄). RBCOD may be sourced, for example, as raw or pretreated (fermented) wastewater, solutions containing volatile fatty acids (VFA) but with negligible bio-available forms of nitrogen and phosphorus. The sources 12 may also be selected residual, process, or wastewater streams containing mixtures of nutrients (nitrogen and phosphorus) as well as RBCOD. By the chosen balance of injection (15), a range of RBCOD:N:P ratios may be established during the accumulation. In one example, source 12A provides a flow of a selected nutrient contained therein via pipe 11A to injector 15A. Injector 15A is commanded to inject the selected nutrient from source 12A into the fed batch reactor. In this case, as shown in FIG. 11, the nutrient is directed via pipe 17A to pipe 17 where the nutrient is blended with any other nutrients commanded by the controller 40 to be injected into the fed batch reactor. In the end, the blended nutrients, including RBCOD, are directed into the reactor via pipe 17.

In the end, the controller 40 is programmed to supply, in one embodiment, determined amounts of phosphorus, nitrogen and/or RBCOD to achieve certain phosphorus to RBCOD and nitrogen to RBCOD ratios as described above. In general, the controller may establish the optimal blend based on the initial conditions of the water quality (RBCOD, nitrogen, phosphorus and other nutrient levels) of the sources, and/or provide for dynamic blending of sources (12) during the accumulation based on the detected (20) water quality trends (RBCOD, nitrogen, phosphorus and other nutrient levels) in the accumulation mixed liquor or the trends of biomass and PHA production in the process (40).

Now turning to FIG. 12, the logic control diagram 200 is shown therein. Logic 200 may be implemented in one of several forms known to those of ordinary skill in control system logic within controller 40 shown in FIG. 11. Logic 200 comprises biomass PHA content comparators 212 and 214, which determine flow of control based on how PHA content of the biomass is being observed to change in the process. Logic 200 also comprises chemical oxygen demand (COD) consumption comparators 216, 222, and 228, which determine flow of control based on how the COD demand from the biomass in the batch is changing. Logic 200 also includes en economic criterion comparator 226, which may determine whether to end the process based on pre-determined economic or other operating conditions. For example, based on experience it may be observed that for a particular type of biomass and size of reactor, it is generally uneconomical to continue the process beyond, for example 24 hours. In one example, comparator 226 would halt the process when the total elapsed process time reached or exceeded 24 hours. Further comprised in logic 200 are control action blocks 218, 220, 224, 230, and 232, which denote the actual control actions commanded by controller 40 when the control passes to each block, respectively.

Once started, block 201, by initiation of a PHA accumulation process, controller 40 may execute this example logic in recurring measurement and control cycles. In each cycle it is determined, as will be described below, what the rates of injection of the various nutrient-bearing materials into the reactor should be to maintain the COD demand of the biomass while adjusting the RBCOD:N or RBCOD:P ratio up or down but generally within the desired range of partial nutrient limitation. Thus in any control cycle it may be determined to increase, hold constant, or decrease any or all of the nutrient flows into the system. The commencement of each measurement and control cycle occurs with the measurement of the content of PHA in the biomass and COD demand based on the COD consumption rate in the batch, block 210. The COD consumption rate may be derived from the combined information of the known supply rate in combination with a measurement of the COD in the maintenance zone. Said amounts may be stored in memory and residing in or interfaced with controller 40 as time series, one time series of PHA content values and one time series of COD demand values. After the first cycle, the change in PHA content and the change in COD demand may be computed by the controller, also illustrated in block 210. It is appreciated that these computations comprise estimating the slopes of the two time series with respect to time. A positive slope for PHA content denotes an increasing PHA accumulation state, and a positive slope of COD demand indicates demand for RBCOD by the biomass. Likewise zero slopes denote constant or steady values of PHA content and of COD demand, respectively, and negative slopes denote decreasing values. It is further appreciated that the slopes may be determined by averaging over a series of preceding cycles for stable operation.

In each cycle, then, after measuring PHA content and COD demand, block 210, a comparator step 212 determines whether PHA content is increasing or not. If PHA is seen to be increasing (positive slope), control passes to comparator 216 where a determination as to whether COD demand is decreasing is made. If COD demand is decreasing (negative slope), then a command to injectors 15 is issued by controller 40 to inject more nutrients and adjust the COD feed rate, and control passes back to block 210 for the next measurement and control cycle. The determination for nutrients to be increased may be made based on maintaining the desired RBCOD:N:P ratio and the measured COD, N, and P. That is, for example, if N is too low relative to COD and P, then an injector among injectors 15A-15D assigned to inject N-bearing material will be commanded by controller 40. As another example, if COD and P are each low relative to N, then injectors assigned to inject RBCOD and P will be activated by controller 40. It appreciated, then, that any of an array of N-bearing materials, P-bearing materials and RBCOD-bearing materials maybe thus blended and injected to bring the RBCOD:N:P ratios in line with the desired ranges.

Returning now to block 216, if the comparison result is that COD demand is not decreasing (not positive), command passed to block 220 where controller 40 either determines that no change in nutrient injection is required, holding nutrient injection rate constant, or that one or more of the nutrient injection rates should be decreased in order to bring the RBCOD:N:P ratios within the desired range. Control then passes back to block 210 for the next measurement and control cycle.

Returning now to comparator 212, if the result of the comparison is that PHA content is not increasing (slope is not positive), then control passes to PHA comparator 214 to determine whether PHA is constant (zero slope) or decreasing (negative slope). In the case of zero slope of the PHA content series, control passes to comparator 228 for determining whether or not COD demand is decreasing (negative slope). If COD demand is decreasing, control passes to block 232 indicating that controller 40 commands increases in injection rates of one or more of the nutrients as has been heretofore described, and control then passes to block 210 for the next measurement and control cycle. If, on the other hand, at block 228 it is determined that COD demand is not decreasing (the COD slope is not negative), then either no changes in nutrient injection rates are commanded by controller 40, as has been heretofore described, or injection rates of one or more of the nutrients is decreased in order to bring the RBCOD:N:P ration within the desired range. Control then passes to block 210 for the next measurement and control cycle.

Returning again to comparator 214, if it is determined that PHA content is not constant, which means in this case that the slope is negative (PHA content is decreasing), then control passes to COD demand comparator 222 for accessing how COD consumption rate is changing. If COD demand is seen not to be decreasing, then excess of nutrients is the case and the injection rates of one or more of the nutrients is decreased in order to bring the RBCOD:N:P rations within range. Control then passes to economic criterion comparator 226. At this point, based on the particular economic or other operating conditions having been met or not, control either passes back to block 210 for the next measurement and control cycle or to block 202 to terminate the process.

Returning to COD demand comparator 222, if COD demand is seen to be increasing, control passes to block 202 terminating the process. That is, decreasing PHA content levels accompanied by increased COD consumption rates may be indicative of having reached a point of diminishing returns with regard to the production of PHA.

### Industrial Embodiments.

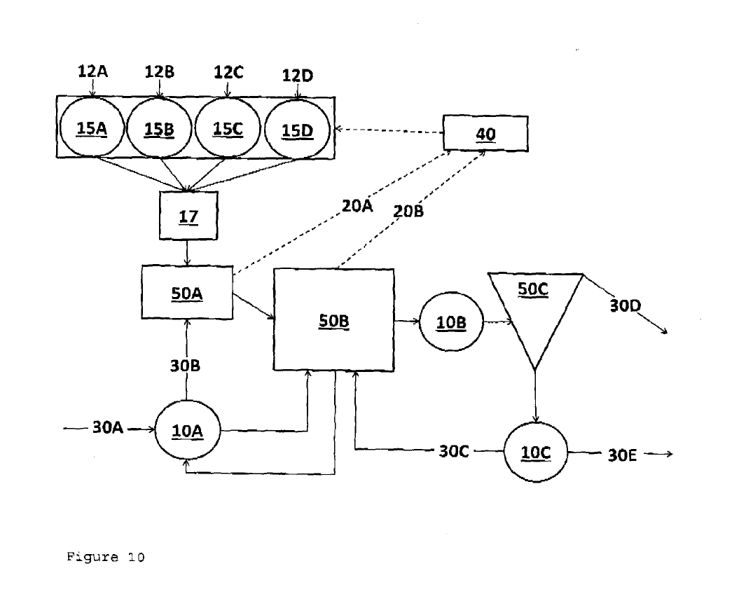

Turning to FIG. 10, an exemplar industrial embodiment of the methods described herein is provided. At the start of the fed-batch process fresh batch of biomass in mixed liquor is delivered via 30A into maintenance zone 50B. At least a fraction of the biomass in mixed liquor is circulated via pump 10A through passage 30B and into stimulating zone 50A. In some embodiments, stimulating zone 50A may be a tank. In some embodiments, the volume of the stimulation zone may be integrated into the volume of the maintenance zone, but be separated from the maintenance zone by a physic& structure. In other embodiments, stimulating zone 50A may be a mixing zone within a tube providing a contact zone for the biomass with RBCOD at an elevated (stimulating) concentration. In yet other embodiments, the stimulation zone is of a sufficient volume to allow the biomass disposed to the stimulation zone a hydraulic retention time of at least twenty seconds.

Various nutrient sources 12A, 12B, 12C, and 12D, to include streams comprising RBCOD, bio-available N, and bioavailable P, are blended together at blending station 17 to form an adjusted feed. Controller 40 controls the amounts of each source 12A, 12B, 12C, and 12D added to the blend through injectors 15A, 15B, 15C, and 15D. Controller 40 determines the amounts to blend from injectors 15A, 15B, 15C, and 15D by analysing data 20A and/or 20B derived from measured values from the stimulating zone 50A and the maintenance zone 50B. Such data includes, inter alia, measurements from which the concentration of RBCOD, N, and/or P may be determined as well as the trends of biomass and PHA production rates. Data from sensors 20A and 20B may also include data sufficient to measure biomass respiration rate. Controller 40 then adjusts injectors 15A, 15B, 15C, and 15D establish a supply rate of RBCOD and such that the N:RBCOD in the adjusted feed is between 2 to 15 mg-N/g-RBCOD and the P:RBCOD in the adjusted feed is between 0.5 to 3 mg-P/g-RBCOD.

After blending at blending station 17, the adjusted feed stream is mixed with the fraction of the mixed liquor containing biomass in the stimulating zone 50A. The fraction is then recycled into the maintenance zone 50B. The mixed liquor in biomass is recycled at such a rate that the average RBCOD concentration in the maintenance zone 50B is less than half the average RBCOD concentration in the stimulating zone 50A. The influent substrate supply rate from 17 and the recycling rate may be determined based on data from sensors 20A and/or 20B. In some embodiments, the recycling rate may be controlled via, inter alia, controller 40.

Biomass in mixed liquor may be removed from the maintenance zone via pump 10D and sent to a settling lank or clarifier 50C. This tank allows the biomass to settle and produces an effluent, which may be removed 30D. The settled biomass may be removed from the settling tank 50C via pump 10C and be recirculated back to the maintenance zone through tube 30C and/or harvested 30E.

It is noted that the some embodiments could combine controller 40, sensors 20A and 20B, sources 12A, 12B, 12C, and 12D, injectors 15A, 15B, 15C, and 15D, and blending station 17 with the systems described in WO 2011-070544 A2. Accordingly, WO 2011/070544 A2 is hereby incorporated in its entirety by reference.

TABLE 1
Water Quality of FWP without any added nutrients

| Parameter | FDS |
|---|---|
| pH | 6.0 |
| COD (g/L) | 33 ± 2 |
| TOC (g/L) | 11 ± 1 |
| N (mg/L) | 74 ± 13 |
| P (mg/L) | 288 ± 17 |

TABLE 2
Water Quality with repect to N/COD and P/COD for PHA Accumulation
with FWP as the source of RBCOD.
FWP RBCOD Feedstock PHA Accumulation Conditions Applied

| | | N/COD | P/COD | Feed | |
|---|---|---|---|---|---|
| Test | Description | (mg/g) | (mg/g) | CODₛₒₗ:Nₛₒₗ:Pₛₒₗ | Feed |
| 1 | Starvation | 0 | 0 | 100:0:0 | VFA mixture |
| 2 | N limitation | 5 | 9 | 100:0.5:0.9 | FWP + NH₄Cl |
| 3 | N limitation | 15 | 9 | 100:1.5:0.9 | FWP + NH₄Cl |
| 4 | N excess | 70 | 9 | 100:7:0.9 | FWP + NH₄Cl |
| Control | N limitation | 2 | 9 | 100:0.2:0.9 | FWP |

TABLE 3
Water Quality with repect to N/COD and P/COD for PHA Accumulation
with Acetate as the source of RBCOD.
Acetate RBCOD Feedstock PHA Accumulation Conditions Applied

| | | N/COD | P/COD | Fed |
|---|---|---|---|---|
| Test | Description² | (mg/g) | (mg/g) | CODₛₒₗ:Nₛₒₗ:Pₛₒₗ |
| 1 | Starvation | 0 | 0 | 100:0.0:0.0 |
| 2 | N limitation, P balance | 2.5 | 9 | 100:0.25:0.9 |
| 3 | N limitation, P balance | 5 | 9 | 100:0.5:0.9 |
| 4 | N & P balance | 50 | 9 | 100:5:9 |
| 5 | N limitation, P limitation | 10 | 0.5 | 100:1.0:0.05 |
| 6 | N limitation, P limitation | 10 | 2 | 100:1.0:0.2 |
| 7 | N limitation, P excess | 10 | 15 | 100:1.0:1.5 |
| Control | N limitation, P balance | 10 | 9 | 100:1.0:0.9 |

TABLE 4
Performances in 20-hour pilot-scale PHA production processes with
acetate as RBCOD, The influence of N/COD and P/COD purposefully
being adjusted was compared to the case of no N and P control.

| | No N and P | N and P |
|---|---|---|
| Parameter | Control | Control |
| Increase in Xₐ (g) | 40.5 | 78 |
| Consumed N (g) | 4.7 | 17.6 |
| Consumed P (g) | No P available | 0.83 |
| Consumed C substrate (g COD) | 907 | 1161 |
| Effective N/COD mg-N/g-COD | 5.2 | 15.2 |
| Effective P/COD mg-P/g-COD | 0.0 | 0.7 |
| PHA content (g-PHA/g-VSS) | 0.29 | 0.38 |
| PHA storage rate q_{PHA} (g-PHA/ | 0.023 | 0.043 |
| g-Xₛ₀ [symbol] h) | | |
| PHA specific production (g-PHA/g-Xₛ₀.) | 0.46 | 0.85 |
| PHA yield, Y_{PHA/S} (g-GOD/g-COD) | 0.28 | 0.25 |
| Active biomass yield, Yₓ [symbol] _{/S} | 0.07 | 0.10 |
| (g-COD/g-COD) | | |

Notes:
The following conversion factors were used: 1 g acetic acid = 1.0667 g COD; 1 g 3-HB = 1.67 g COD; 1 g VSS = 1.45 g COD; 1 g Xa =1.45 g COD, where Xa corresponds to non-PHA VSS (X [symbol] = VSS − PHA).
X [symbol] 0 refers to the initial active biomass supplied to the fed-batch accumulation process.
S refers to RBCOD as substrate. [symbol]
indicates data missing or illegible when filed

What is claimed is:

1. A feed-on-demand fed-batch method of producing a PHA-rich-biomass from open mixed cultures, comprising:
directing mixed liquor containing biomass into a fed-batch reactor including at least one biomass stimulating zone and at least one biomass maintenance zone;
providing a feed containing readily biodegradable chemical oxygen demand (RBCOD) along with bio-available nitrogen (N) and phosphorus (P);
adjusting the concentration of the bio-available N relative to the concentration of RBCOD to yield an adjusted feed with an average N to RBCOD ratio within the range of 2 to 15 mg-N/g-RBCOD;
adjusting the concentration of the bio-available P relative to the concentration of RBCOD to yield an adjusted feed with an average P to RBCOD ratio within the range of 0.5 to 3 mg-P/g-RBCOD;
intermittently and repeatedly stimulating the biomass respiration rate for at least a fraction of the biomass in the reactor by exposing the fraction of the biomass to the adjusted feed in the stimulating zone such that the biomass is stimulated on average to greater than 50% of its extant maximum respiration rate;
after exposing the fraction of the biomass to the adjusted feed in the stimulating zone, transferring at least a portion of the fraction of biomass from the stimulating zone to the maintenance zone where the average RBCOD concentration is less than half the average concentration of RBCOD in the stimulating zone; and
circulating the mixed liquor containing biomass back and forth between the stimulating zone and the maintenance zone such that fractions of biomass are repeatedly exposed in order to reach a high respiration rate with the adjusted feed supplied to the stimulating zone and wherein the biomass is otherwise maintained at a lower RBCOD concentration in the maintenance zone.

2. The method of claim 1, further comprising supplying the adjusted feed to the stimulating zone at a rate that is proportional to the demand created by the biomass respiration rate.

3. The method of claim 1 wherein the feed is blended from at least two distinct sources, to provide a selected concentration of nitrogen with respect to RBCOD in the feed supplied to the stimulation zone(s).

4. The method of claim 1 wherein the feed is blended from at least two distinct sources, to provide a selected concentration of phosphorus, with respect to RBCOD in the feed supplied to the stimulation zone(s).

5. The method of claim 1 including measuring the phosphorus, nitrogen, and/or RBCOD concentrations in, and/or supplied to, the fed-batch reactor and in response to the measured concentrations, adjusting the concentration of nitrogen, phosphorus and/or RBCOD in the feed supplied to the stimulation zone(s).

6. The method of claim 1 including measuring the biomass PHA content and/or the time rate of change of the biomass PHA content for the biomass in the fed-batch reactor and in response to the measured content or changes in content with time, adjusting the concentration of nitrogen, phosphorus and/or RBCOD established in the feed supplied to the stimulation zones(s).

7. The method of claim 1 including measuring the biomass respiration rate and/or the time rate of change of the biomass respiration rate for the biomass in the fed-batch reactor, and in response to the measured respiration rate or respiration rate changes in time, adjusting the concentration of nitrogen, phosphorus or RBCOD in the feed supplied to the stimulation zone(s).

8. The method of claim 1 including measuring the consumption rate of COD and/or the time rate of change of the COD consumption rate by the biomass in the fed-batch reactor, and in response to the measured COD consumption rate or the COD consumption rate changes in time, adjusting the concentration of nitrogen, phosphorus or RBCOD in the feed supplied to the stimulation zone(s).

9. The method of claim 1 including a combination of at least two of the methods of 6, 7 and 8.

10. The method of claim 1 wherein the fed-batch reactor includes at least two tanks with at least one tank including the stimulation zone and at least one tank including the maintenance zone and wherein the feed to the fed-batch reactor is directed to the tank that includes the stimulating zone.

11. The method of claim 1 wherein the volume of the stimulation zone is sufficiently large to provide the biomass disposed to the stimulation zone a hydraulic retention time of at least 20 seconds.

12. The method of claim 1 wherein the volume of the stimulation zone is integrated into the volume of the maintenance zone, but where the volume of the stimulation zone is at least partly separated from the maintenance zone by a physical structure.

13. The method of claim 1 including biologically treating a wastewater stream In a biological wastewater treatment system and separating the mixed liquor and biomass from the wastewater stream and directing the mixed liquor and biomass to the fed-batch reactor.

14. The method of claim 1 including continuing to circulate the mixed liquor containing the biomass back and forth between the stimulating zone and the maintenance zone and continuing to maintain the phosphorus and nitrogen to RBCOD ratios within said range until the PHA content of the biomass is, on average, greater than 0.40 g-PHA/g-VSS.

15. The method of claim 1 including harvesting at least a fraction of the biomass from the process during the fed-batch operation and before the batch process terminates.

16. The method of claim 15 including starting to harvest at least a fraction of the biomass from the process during the fed-batch operation once the biomass PHA content is greater than 0.40 g-PHA/g-VSS.

17. The method of claim 1 wherein stimulating the biomass respiration rate is such that the biomass is stimulated, on average, to greater than 70% of its extant maximum respiration rate.

18. The method of claim 1 wherein the biomass is harvested from a biological wastewater treatment process prior to being directed to the fed-batch reactor and wherein the PHA content of the harvested biomass is less than 10% of the dry biomass weight.

19. The method of claim 1 including pretreating the mixed liquor prior to directing the mixed liquor into the fed-batch reactor wherein pretreating the mixed liquor includes thickening the mixed liquor so as to reduce the concentrations of phosphorus and nitrogen in the mixed liquor directed to the fed-batch reactor.

20. The method of claim 19 wherein after thickening the mixed liquor, adding a dilutent to the mixed liquor to further reduce the concentrations of phosphorus and nitrogen in the mixed liquor.

21. A feed on demand fed-batch method for producing a PHA-rich-biomass from open mixed cultures, comprising:
directing mixed liquor containing biomass into a fed-batch reactor including at least one biomass stimulating zone and at least one biomass maintenance zone;
directing a feed into the fed-batch reactor where the feed contains RBCOD along with bioavailable N and P;
intermittently and repeatedly stimulating the respiration rate of at least a portion of the biomass by directing the feed into the fed-batch reactor and exposing the biomass to a relatively high concentration of RBCOD in the stimulating zone;
after the biomass has been exposed to the relatively high concentration of biomass in the stimulating zone, transferring the biomass to the maintenance zone where the biomass is exposed to a relatively low concentration of RBCOD;
circulating the biomass back and forth between the stimulating zone and the maintenance zone; and
controlling the N or P concentration relative to the RBCOD in the feed directed into the fed-batch reactor by controlling the N to RBCOD ratio or the P to RBCOD ratio in the feed.

22. The method of claim 21 including controlling the N and P concentrations relative to the RBCOD in the feed by adjusting the concentration of the bioavailable N relative to the concentration of RBCOD to yield an adjusted feed with an average N to RBCOD ratio within a range of 2 to 15 mg-N/g-RBCOD and the concentration of bioavailable P relative to the concentration of RBCOD to yield a feed having an average P to RBCOD ratio within the range of 0.5 to 3 mg-P/g-RBCOD.

23. The method of claim 21 including measuring the N and P concentrations in the mixed liquor in the fed-batch reactor and adjusting the N to RBCOD ratio or the P to RBCOD ratio in the feed to yield selected ratios within a pre-determined range.

24. The method of claim 21 including measuring signals that infer changes in the biomass PHA content, biomass respiration rate and/or the COD consumption rate in the fed batch reactor and adjusting the N to RBCOD ratio or the P to RBCOD ratio in the feed to yield selected ratios within a pre-determined range based on one or more of these signals.

25. The method of claim 23 wherein sensors are utilized to measure N, P and RBCOD in the mixed liquor in the fed-batch reactor or in the feed and the sensors are operative to direct data to a controller where the data is representative of the concentrations of P, N, and/or RBCOD, and wherein the controller is operative to control the ratio of N to RBCOD and P to RBCOD such that on average the ratios fall within a pre-selected range.

26. The method of claim 24 wherein sensors are utilized to measure or infer proportional changes in biomass PHA content, biomass respiration rate, and/or COD consumption rate and the sensors are operative to direct data to a controller where the data is representative of the concentrations of P, N, and/or RBCOD in the feed supplies, and wherein the controller is operative to control the ratio of N to RBCOD and P to RBCOD such that on average the ratios fall within a pre-selected range.

27. The method of claim 21 including controlling the N concentration relative to the RBCOD in the feed by adjusting the concentration of the bioavailable N relative to the concentration of RBCOD to yield an adjusted feed with an average N to RBCOD ratio within a range of 2 to 15 mg-N/g-RBCOD.

28. The method of claim 21 including controlling the P concentration relative to the RBCOD in the feed by adjusting the concentration of bioavailable P relative to the concentration of RBCOD to yield a feed having an average P to RBCOD ratio within the range of 0.5 to 3 mg-P/g-RBCOD.

29. The method of claim 21 including measuring the biomass PHA content and/or the time rate of change of the biomass PHA content for the biomass in the fed-batch reactor and in response to the measured content or changes in content with time, adjusting the concentration of nitrogen, phosphorus and/or RBCOD established in the feed supplied to the stimulation zones(s).

30. The method of claim 21 including measuring the biomass respiration rate and/or the time rate of change of the biomass respiration rate for the biomass in the fed-batch reactor, and in response to the measured respiration rate or respiration rate changes in time, adjusting the concentration of nitrogen, phosphorus or RBCOD in the feed supplied to the stimulation zone(s).

31. The method of claim 21 including continuing to circulate the mixed liquor containing the biomass back and forth between the stimulating zone and the maintenance zone and continuing to maintain the phosphorus and nitrogen to RBCOD ratios within said range until the PHA content of the biomass is, on average, greater than 0.40 g-PHA/g-VSS.

32. The method of claim 21 wherein stimulating the biomass respiration rate is such that the biomass is stimulated, on average, to greater than 70% of its extant maximum respiration rate.
